(19) United States
(12) Patent Application Publication
(10) Pub. No.: US 2023/0227511 A1
(43) Pub. Date: Jul. 20, 2023

(54) THERAPEUTIC OR PREVENTIVE AGENT FOR INFECTIOUS DISEASE

(71) Applicant: NIPPON MEDICAL SCHOOL FOUNDATION, Tokyo (JP)

(72) Inventors: Takao HAMAKUBO, Tokyo (JP); Kenji DAIGO, Tokyo (JP); Keita SODA, Tokyo (JP)

(73) Assignee: NIPPON MEDICAL SCHOOL FOUNDATION, Tokyo (JP)

(21) Appl. No.: 17/924,204

(22) PCT Filed: May 11, 2021
(86) PCT No.: PCT/JP2021/017849

§ 371 Date: Nov. 9, 2022

(30) Foreign Application Priority Data
May 14, 2020 (JP) 2020-084916

(51) Int. Cl. C07K 14/435; A61P 31/00

(52) U.S. Cl. CPC C07K 14/435; A61P 31/00; C07K 2319/30

(57) ABSTRACT

It is an object of the preset invention to provide a usefultool for treating or preventing infections disease. According to the present invention, provided is a therapeutic or preventive agent for infections disease, comprising, as an active ingridient, a fusion protein of (a) at least one polypeptide comprising an amino acid sequence that is identical to or substantially identical to the amino acid sequence of the N-terminal domain of pertraxin 3 capable of binding to a bistone to form a polypeptide aggregate and (b) the Fe portion of an immunoglobulin, or a pharmacologically acceptable salt thereof.

### TECHNICAL FIELD

The present invention relates to a therapeutic or preventive agent for infectious disease.

### BACKGROUND ART

Pentraxin 3 (PTX3) is a pauern-recognizing molecule that belongs to the pentraxin family. The pentraxin family is a generic name for proteins having a common pentraxin domain on the C-terminal side, and based on the characteristies of the primary structure, the pentraxin is classified into two types, namely, short pentraxin and long pentraxin. C-reactive protein (CRP), serum amyloid P component (SAP) and the like belong to the short pentraxin, whereas PTX3 belongs to the long pentraxin. The primary structure of PTX3 is constituted with an N-terminal domain (18 to 178 amino acids) and a pentraxin domain on the C-terminal side (179 to 381 amino acids), which are longer than the short pentraxin, and its higher structure forms an octamer via a disulfide bond.

Differing from CRP and SAP that are generated in the liver, PTX3 is expressed in a wide variety of cell species due to inflammatory signals, and is characterized in that it shows a topical expression pattern. As a characteristic generation mechanism of PTX3 PTX3 stored in neutrophil graves is released into the outside of the cells by stimulation of pathogens or Toll-like receptor (TLR) agonists. The released PTX3 functions as a constitutional protein of structures for trapping and killing pathogens, consisting of DNAs and a group of antibacterial proteins, which are called “Neutrophil extracellular traps (NETs)” (Non-Patent Document 1). It has been reported that PTX3 has a wide variety of functions in a living body, and that, for example, PTX3 plays a role in inflammatory regulation, innate immune response, pregnancy maintenance, etc. (Non-Patent Document 2). PTX3 also has the function of binding to a large number of proteins, and exhibits specific functions cooperatively with the bound proteins.

It has been reported that the blood concentration of PTX3 increases in various infectious diseases (Non-Patent Document 4). It has been known that, in particular, in sepsis, the concentration of PTX3. which is generally 2 ng/mL or less, increases to approximately 200 to 800 ng/mL, and that the concentration of PTX3 correlates with the survival percentage (Non-Patent Document 3). Moreover, it has also been reported that PTX3 transgenic mice are resistant to death due to sepsis (Non-Patent Document 4).

The present inventors have found that the activity of PTX3 to bind (aggregate) to a histone and to suppress the cytotoxicity of the histone is not present in the C-terminal domain of PTX3. but is sufficiently present in the N-terminal domain of PTX3. The present inventors have further confirmed that a 50-amino acid peptide in the N-terminal domain of PTX 3 has an action to suppress cytotoxicity (Patent Document 1).

It has been considered that PTX3 directly binds to various pathogens and triggers a defense reaction. Also regarding viruses, it has been known that PTX3 directly binds to influenza virus, coronavirus (SARS-CoV and mouse hepatitis virus (MHV)), etc.

Coronavirus has a spike protein on the surface thereof, and this virus recognizes angiotensin-converting enzyme 2 (ACE2) on a host cell membrane and binds thereto, so that the host is infected with the coronavirus. SARS-CoV-2, which has caused a pandemic in 2020, has a spike protien comprising an amino acid sequence that is highly homologous to those of previous SARS virus or MERS virus, and SARS-CoV-2 has a strong binding ability to ACE2. It has been known that the antibody reacting against this spike protein includes a neutralizing antibody that inhibits the binding of virus to ACE2 and inhibits the infection with the virus.

Moreover, it has been pointed out that COVID-19 causes pneumonia, and in a case where COVID-19 becomes severe, it affects vascular endothelial cells and causes vasculitis attended with cytokine storm, thrombosis and further, multiple organ failure, which may lead to death (Non-Patent Documents 5 and 6). Although detailed mechanisms have not yet been known, it is conceived that mutation of the nucleocapsid protein of SARS-CoV-2 virus correlates with the disease that becomes severe and that thrombosis or endothelial cell dysfunction caused by NETs (Neutrophil extracellular traps) released from neutrophils is also a mechanism for the disease that becomes severe, which becomes a target of a new drug (Non-Patent Document 7).

### PRIOR ART DOCUMENTS

### Non-Patent Documents

Non-Patent Document 1 Jaillon. S., et al. (2007) J Exp Med 204. 793-804
Non-Patent Document 2: Mantovani, A., et al (2008) J Clin immunal 28, I-13
Non-Patent Document 3: Mauri, T., et al. (2010) Intensive Care Med 36, 621-629
Non-Patent Document 4: Dias A. A., et al. (2001) J Leukoe Biol 69., 928-836
Non-Patent Document 5: Barnes BJ et al, JEM 217-6, 2020
Non-Patent Document 6: Libby, P and Luscher T. European Heart Journal 41, 3038-3044. 2020
Non-Patent Document 7: Narasanaju T et al., Frontiers in Pharmacology, 11: 870. 2020

### Patent Documents

Patent Document 1: WO/2013/191280

### SUMMARY OF INVENTION

### Object to Be Solved by the Invention

PTX3 is mainly present in neutrophile and upon inflammation, it is released as a part of neutrophil extracellular traps (NETs) and exhibits antibacterial action, complement activation action, optimization action, and the like. The present inventors had previously detected the binding of PTX3 with a histone, and had found that, in sepsis, PTX3 suppresses the cytotoxic action of extracellular histones released from neutrophils and the like against vascular endothelial cells (Patent Document 1). PTX3 is a protein having a molecular weight of approximately 40,000, which binds to a plurality of proteins in blood to form an enrmous complex. It had been known that it is difficult to prepare a formulation from a protein having a large molecular weight, and that PTX3 is an easily aggregated protem and thus, is hardly adjusted. Hence, in Patent Document 1, it had been attempted to identify a partial peptide having activity, and as a result, it had been found that a 50-amino acid peptide on the N-terminal side has an action to suppress histone cytotoxicity against vascular endothelial cells (Patent Document 1).

Thereafter, the present inventors have administered the 50-amino acid peptide (PTX3N50) to mouse models. However, the present inventors could not improve the survival percentage of the sepsis mouse models. From these results it has been considered that the peptide is problematic in terms of stability in blood, etc. It is an Object of the present invention to provide a useful tool for treating or preventing infectious disease. Further, it is another object of the present invention to show that PTX3 directly binds to a protein that is associated with infection with SARS-CoV-2 as a causative virus of COVID-19 infection, further to identify the sequence of PTX3 associated with the aforementioned binding, and further to provide an anti-infection agent, a therapeutic agent, etc.

### Means for Solving the Object

As a result of intensive studies conducted directed towards achieving the aforementioned objects, the present inventors have found that a significant life-extendmg effect could be obtained in sepsis mouse models by converting a peptide in the N-terminal domain of PTX3 to the form of an Fc fusion. Moreover, the present inventors have shown that PTX3 associated with innate immune response binds to a spike protein associated with infection with coronavirus, have discovered an important partial peptide of PTX3 associated with the aforementioned binding, and have then demonstrated that it is possible to design a molecule that regulates the infection. Furthermore, the present inventors have demonstrated that the nucleocapsid protein of coronavirus impairs vascular endothelial cells, as with histones comprised in NETs, and have further demonstrated that the partial peptide PTX3 has an action to suppress the impairment by the nucleocapsid protein of coronavirus. Thus, the present inventors have demonstrated that the PTX3-derived peptide is able to suppress the infection with coronavirus and to prevent the infections disease from becoming severe. The present invention has been completed based on the above-described findings.

Specifically, the present invention relates to the following features.

<1> A therapeutic or preventive agent for infectious disease, comprising, as an active ingredient, a fusion protein of (a) at least one polypeptide comprising an amino acid sequence that is identical to or substantially identical to the amino acid sequence of the N-terminal domain of pentraxin 3 capable of binding to a histone to form a polypeptide aggregate and (b) the Fe portion of an immunoglobulin, or a pharmacologically acceptable salt thereof.

<2> The therapeutic or preventive agent according to <1>, wherein the amino acid sequence of the N-terminal domain of the pentraxin 3 is a partial sequence consisting of consecutive 15 or more amino acids of the amino acid sequence as set forth in SEQ ID NO: 2.

<3> The therapeutic or preventive agent according to <1> or <2>, wherein the amino acid sequence of the N-terminal domain of pentraxin 3 is a partial sequence consisting of consecutive 15 or more amino acids in the amino acid sequence consisting of the 1st to 120th amino acids of the amino acid sequence as set forth in SEQ ID NO: 2.

<4> The therapeutic or preventive agent according to any one of <1> to <3>, wherein the amino acid sequence of the N-terminal domain of pontraxin 3 comprises any one of the following regions:
(1) the region consisting of the 18th to 67th amino acids of the amino acid sequence as set forth in SEQ ID NO: 2,
(2) the region consisting of the 18th to 37th amino acids of the amino acid sequence as set forth in SEQ ID NO: 2,
(3) the region consisting of the 38th to 57th amino acids of the amino acid sequence as set forth in SEQ ID NO: 2,
(4) the region consisting of the 38th to 77th amino acids of the amino acid sequence as set forth SEQ ID NO: 2,
(5) the region consisting of the 78th to 97th amino acids of the amino acid sequence as set forth in SEQ ID NO: 2,
(6) the region consisting of the 98th to 117th amino acids of the amino acid sequence as set forth in SEQ ID NO: 2.
(7) the region consisting of the 85th to 144th amino acids of the amino acid sequence as set forth in SEQ ID NO: 2, and
(8) the region consisting of the 119th to 176th amino acids of the amino acid sequence as set forth in SEQ ID NO: 2.

<5> The therapeutic or preventive agent according to any one of <2> to <4>, wherein at least one cysteine residue of the amino acid sequence as set forth in SEQ ID NO: 2 is substituted with another amino acid residue.

<6> The therapeutic or preventive agent according to any one of <2> to <5>, wherein the cysteine residues that are the 47th and 49th amino acid residues of the amino acid sequence as set forth in SEQ ID NO: 2 are substituted with other amino acid residues,

<7> The therapeutic or preventive agent according to <5> or <6>, wherein the other amino acid residues are serine residues.

<8> The therapeutic or preventive agent according to any one of <1> to <7>, wherein (b) the N-terminus of the immunaglobulin Fe portion is fused to the C-terminus of (a) the polypeptide comprising an amino acid sequence that is identical to or substantially idnetical to the amino acid sequence of the N-terminal domain of pentraxin 3 capable of binding to a histone to form a polypeptide aggregate.

<9> The therapeutic or preventive agent according to any one of <1> to <8>, wherein the infectious disease is sepsis.

<10> The therapeutic or preventive agent according to any one of <1> to <8>, wherein the infectious disease is COVID-19 infection.

### Advantageous Effects of Invention

According to the present invention, infectious disease can be treated or prevented by administering a fusion protein of the N-terminal domain of pentraxin 3 and Fe to a patient.

### BRIEF DESCRIPTION OF DRAWINGS

FIG. 1 shows a design of PTX3-Fe fusion proteins.

FIG. 2 shows the effects of PTX3-Fe fusion proteins to suppress histone cytotoxicity against vascular endothelial cells,

FIG. 3 shows the improvement of the survival percentage of sepsis mouse models by a PTX3-Fe fusion protein.

FIG. 4 shows the effects of the 5 types of PTX3 20-amino acid peptides (100 ug/ml) to suppress the cytotoxicity of histone H3 (100 ug/ml).

FIG. 5 shows the effects of the 5 types of PTX3 20-amino acid peptides (100 ug/ml) to suppress the cytotoxicity of histone H4 (100 ug/ml).

FIG. 6 shows a design of PTX3-Fe fusion proteins each comprising 60 amino acids.

FIG. 7 shows the results of an experiment regarding the binding of various types of 60. amino acid PTX3-Fe fusion proteins with the spike protein (SPN-C52H8)

FIG. 8 shows the cytotoxic activity of a corona virus nucleocapsid protein against vascular endothelial cells (HUVEC). H2O: water: Opti-MEM (HUVEC culture solution): HCoV-NL63: virus nucleocapsid (N) protein: SARS-CoV: virus N protein: SARS-CoV-2: virus N protein.

FIG. 9 shows the results of an experiment regarding the binding of various types of 60-amino acid PTX3-Fe fusion proteins with the N protein (NP) of SARS-CoV2.

FIG. 10 shows the effects of 60PTX3-Fe proteins to suppress the cytotoxic activities of the N proteins (NP) of Sars-CoV-2 against HUVEC.

### EMBODIMENTS OF CARRYING OUT THE INVENTION

Hereinafter, the embodiments of the present invention will be described.

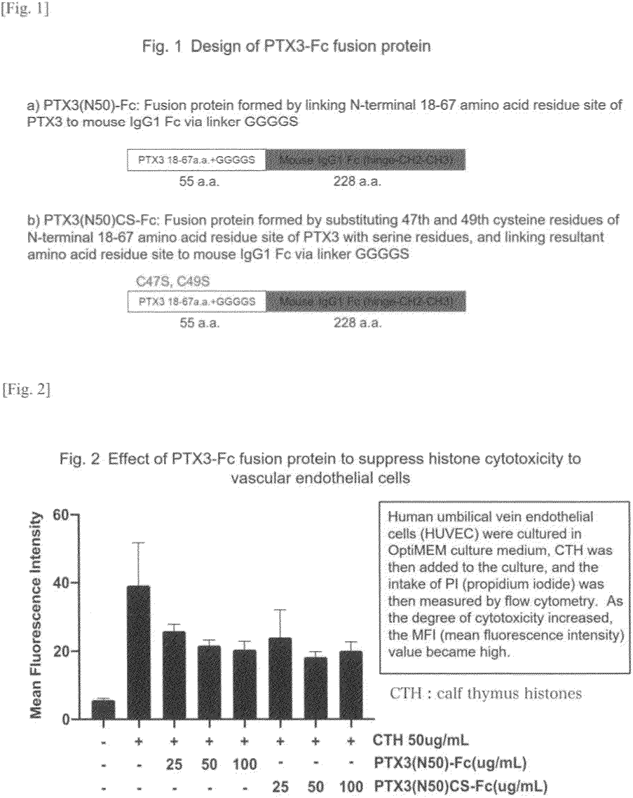

In the Eamples of the present invention, CHO recombinant cells were produced, and thereafter, a PTX3-Fe fusion protein (PTX3(N50)-Fe), in which a peptide consisting of 50 amino acid residues on the N-terminal side of PTX3 was fused to the Fe portion of an immunoglobulin, and an ammo acid mutant thereof, were purified from the culture solution by affinity column chromatography (FIG. 1). PTX3(N50)-Fe and the amino acid mutant thereof bound to histones, and suppressed the cytotoxicity of the histones against vascular endothelial cells (FIG. 2). Moreover, in an experiment using sepsis mouse models administered with LPS, it was confirmed that both PTX3(N50)-Fe and the amino acid mutant thereof had significant life-extending effects, and further, it was revealed that the amino acid mutant had a higher life-extending effect than PTX3(N50)-Fe. As described above, it was demonstrated that the PTX3-Fe fusion protein (PTX3(N50)-Fe) and the amino acid mutant thereof are useful as therapeutic of preventive agents for infectious diseases such as sepsis.

The therapeutic or preventive agent for infectious disease of the present invention comprises, as an active ingredient, a fusion protein of:
(a) at least one polypeptide comprising an amino acid sequence that is identical to or substantially to the amino acid sequence of the N-terminal domain of pentraxin 3 capable of binding to a histone to form a polypeptide aggregate: and
(b) the Fe portion of an immunoglobulin, or
a pharmacologically acceptable salt thereof

Hereinafter, the polypeptide comprising an amino acid sequence that is identical to or substantially identical to the amino acid sequence of the N-terminal domain of pentraxin 3 capable of binding to a histone to form a polypeptide aggregate may be also referred to as “the polypeptide used in the present invention” at times.

PTX3 is a known protein belonging to a protest family generally called “pentraxin family,” and among others, PTX3 is a protein belonging to long pentraxin. The PTX3 of the present invention is generally derived from vertebrates.

Examples of the vertebrates may include mammals, Aves, fishes, amphibians, and reptiles. The mammals are not particularly limited, and examples of the mammals may include experimental animals including rodents such as mice, rats, hamsters and Guinea pigs, and rabbits; livestock animals such as pigs, bovines, goats, horses, sheep, and minks; companion animals such as dogs and cats: and primates such as humans, monkeys, rhesus monkeys, marmosets, orangutans, and chimpanzees. Examples of the Aves may include chickens, quails, dicks, gooses, turkeys, ostriches, emus, Struthio camelus, guinea fowls, and pigeons. The vertebrates are preferably mammals, and more preferably humans.

In the present description when a polypeptide or a polynucleotide is described with the term “organism X-derived,” it means that the amino acid sequence of the polypeptide of the nucleic acid sequence of the polynucleotide is identical to the amino acid sequence of the polypeptide or the nucleic acid sequence of the polynucleotide, which is naturally expressed in the organism X.

Human-dervied PTX3 is representatively consists of a single-stranded polypeptide having an entire length of 381 amino acids. A representative amino acid sequence of the human-derived PTX3 polypeptide has been registered under GenBank Accession No. AAH39733 (SEQ ID NO: 2). In addition, a representative nucleotide sequence encoding the human-derived PTX3 polypeptide has been registered under GenBank Accession No. BC039733 (SEQ ID NO: 1).

In general, regarding a PTX3 polypeptide expressed in a cell, the signal peptide thereof at the N-tenninus was cleaved in a process in which the PTX3 polypeptide is secreted to the outside of the cell, and thus, the PTX3 polypeptide becomes a mature PTX3 polypeptide. In the present description, the PTX3 polypeptide is preferably a mature PTX3 polypeptide. For example, in the amino acid sequence of human-derived PTX3, a portion consisting of the 1st to 17th amino acids counted from the N-terminus is a signal peptide, and this signal peptide is cleaved in a process in which the PTX3 polypeptide is secreted to the outside of the cell and then becomes a mature polypeptide Accordingly, a human-derived mature PTX polypeptide representatively comprises the amino acid sequence consisting of the 18th to 381st amino acids of the amino acid sequence as set forth in SEQ ID NO: 2,

In the present invention, the N-terminal domain of PTX3 is a region that is located closer to the N-terminal side than the pentraxin domain of the aforementioned PTX3 polypeptide (preferably, a mature PTX3 polypeptide), or a part of the region. The pentraxin domain is a domain that is common in the members of the pentraxin superfamily, such as CRP (C-reactive Protein) and SAP (Serum amyloid P component, and the pentraxin domain has been registered with the Conserved Domain of NCBI under Accession No. cd00152. Accordingly, a person skilled in the art could specify the pentraxin domain based on the sequence information of any given PTX3, and could also specify the N-terminal domain or the PTX3. The pentraxin domain of human-derived PTX3 representatively corresponds to the region consisting of the 179th to 380th amino acids of the amino acid sequence as set forth in SEQ ID NO: 2. Therefore, the N-terminal domain of human-derived PTX3 is generally the region consisting of the 1st to 178th amino acids of the amino acid sequence as set forth in SEQ ID NO: 2, or a part thereof, and is preferably the region consisting of the 18th to 178th amino acids of the amino acid sequence as set forth in SEQ ID NO: 2, or a part thereof. In the case of human-derived PTX3, the N-terminal domain thereof is preferably the portion consisting of the 18th to 178th amino acids counted from the N-terminus. The amino acid sequence of the region consisting of the 18th to 178th amino acids of the amino acid sequence as set forth in SEQ ID NO: 2 is as set forth in SEQ ID NO: 3.

When the N-temrinal domain of PTX3 is a part of the region that is located closer to the N-tenninal side than the pentraxin domain of the PTX3 polypeptide, the length of the N-terminal domain of PTX3 is not particularly limited, as long as it has the activity of binding to a histone to form a polypeptide aggregate. The length of the part is at least 8 amino acids, tor example, 10 amino acids or more, preferably 15 amino acids or more, and more preferably 20 amino acids or more. The length of the amino acids may also be 30 amino acids of more, or 50 amino acids or more.

The amino acid sequence of the N-terminal domain of PTX3 is preferably a partial sequence consisting of at least consecutive 8 amino acids (for example, 10 amino acids or more, preferably 15 amino acids or more, and more preferably 20 amino acids or more) in the amino acid sequence consisting of the 1st to 120th amino acids of the amino acid sequence as set forth in SEQ ID NO: 2.

The amino acid sequence of the N-terminal domain of PTX3 is preferably an amino acid sequence consisting of the Xth amino acid sequence as set forth in SEQ ID NO: 2. In this context, X is preferably 18, 19, 20, 21, 22, 23, 24, 25, 26, 27, 28, 29, 30, 31, 32, 33, 34, 35, 36, 37, 38. 39, 40, 41, 42, 43, 44, 245, 46, or 47, whereas Y is preferably 87. 86, 85, 84, 83, 82, 81, 80, 79, 78, 77, 76, 75, 74, 73, 72, 71, 70, 69, 68, 67, 66, 65, 64, 63, 62, 61, 60, 59, or 58.

In one example of the present invention, the amino acid sequence of the N-terminal domain of PTX3 comprises any one the following regions:
(1) the region consisting of the 18th to 67th amino acids of amino acid sequence as set forth in SEQ ID NO: 2,
(2) the region consisting of the 18th to 37th amino acids of the amino acid sequence as set forth in SEQ ID NO: 2,
(3) the region consisting of the 38th to 57th amino acids of the amino acid sequence as set forth in SEQ ID NO: 2,
(4) the region consisting of the 58th to 77th amino acids of the amino acid sequence as set forth in SEQ ID NO: 2,
(5) the region consisting of the 78th to 97th amino acids of the amino acid sequence as set forth in SEQ ID NO: 2,
(6) the region consisting of the 98h to 117th amino acids of the amino acid sequence as set forth in SEQ ID NO: 2,
(7) the region consisting of the 85th to 144th amino acids of the amino acid sequence as set forth in SEQ ID NO: 2, and
(8) the region consisting of the 119th to 176th amino acids of the amino acid sequence as set forth in SEQ ID NO: 2.

The polypeptide used in the present invention comprises an amino acid sequence that is identical to or substantially identical to the N-terminal domain of PTX3 Such an amino acid sequence that is substantially identical to the amino acid sequence of the N-terminal domain of PTX3 may be an amino acid sequence having an identity of 50% or more, preferably 60% or more, more preferably 70% or more, even more preferably 80% or more, further preferably 90% or more, particularly preferably 95% or more, and most preferably 99% or more, to the amino acid sequence of the N-terminal domain of PTX3. In this context, the term “identity” means the percentage of identical amino acids to all amino acid residues to be overlapped, in a case where two amino acid sequences are aligned by using a mathematical algorithm known in the present technical field.

Examples of such an amino acid sequences that is substantially identical to the amino acid sequence of the N-terminal domain of PTX3 may include (1) an amino acid sequence comprising a deletion of 1 or 2 or more amino acids (preferably about 1 to 30, more preferably about 1 to 10, and further preferably 1 or 2amino acid sequence of the N-terminal domain of PTX3; (2) an amino acid sequence comprising an addition of 1 or 2 or more amino acids (preferably about 1 to 30, more preferably about 1 to 10, and further preferable 1 or 2 amino acids) to the amino acid sequence of the N-terminal domain of PTX3; (3) an amino acid sequence comprising an insertion of 1 or 2 or more amino acids (preferably about 1 to 30, more preferably about 1 to 10, and further preferably 1 or 2 amino acids) into the amino acid sequence of the N-terminal domain of PTX3; (4) an amino acid sequence comprising a substitution of 1 or 2 or more amino acids (preferably about 1 to 30, more preferably about 1 to 10, and further preferably 1 or 2 amino acids) in the amino acid sequence of the N-terminal domain of PTX3, with other amino acids: and (5) an amino acid sequence comprising a combination thereof.

As described above, when an amino acid sequence is subjected to an insertion, deletion, addition or substitution, the position in which such an insertion, deletion, addition or substitution takes place is not particularly limited, as long as a polypeptide having the amino acid sequence has the activity of binding to a histone to form a polypeptide aggregate. Examples of the amino acid sequence that is substantially identical to the amino acid sequence of the N-terminal domain of PTK3, which can be used in the present invention, may include the amino acid sequences of homologs in vertebrates other than the aforementioned human.

In the present invention, at least one cysteine residue of the amino acid sequence as set forth in SEQ ID NO: 2 is preferably substituted with another amino acid residue. More preferably, cysteine residues that are the 47th and 49th amino acid residues of the amino acid sequence as set forth in SEQ ID NO: 2 are substituted with other amino acid residues. Such other amino acid sequences are preferably serine residues.

The polypeptide comprising the amino acid sequence that is substantially identical to the amino acid sequence of the N-terminal domain of PTX3 is preferably a polypeptide comprising an amino acid sequence that is substantially identical to the above-described amino acid sequence of the N-terminal domain of PTX3. and having an activity that is substantially homogeneous to that of the polypeptide comprising the amino acid sequence of the N-terminal domain of PTX 3.

The substantially homageneous activity may be, for example, the activity of binding to a histone to form a polypeptide aggregate. In this context, the term “substantially homogeneous” means that the properties are qualitatively (e.g. physiologically or pharmacologically) homogeneous. Accordingly, the activities of the above-described polypeptides consisting of amino acid sequences that are substantially identical to each other are preferably equivalent to each other. However, the polypeptides may be ditferent from each other, in terms of the degree of the activity (for example, about 0.01 to about 100 times, preferably about 0.1 to about 10 times, and more preferably 0.5 to 2 times) and quantitative factors such as the molecular weights of the polypeptides.

The length of polypeptide used in the present invention is not particularly limited, as long as the polypeptide has the activity of binding to a histone to form a polypeptide aggregate. From the viewpoint of the ease of preparation and the stability of the polypeptide, the length of the present polypeptide is, for example, 200 amino acids or less, preferably amino acids or less, more preferably 50 amino acids or less, and further preferably 30 amino acids or less.

Examples of the polypeptide used in the present invention may include the amino acid sequence consisting of the 18th to 67th amino acids of the amino acid sequence as set forth in SEQ ID NO: 2, and an amino acid sequence in which the cysteine residues that are the 47th and 49th amino acid residues in the aforementioned amino acid sequence are substituted with other amine acid residues (e.g. serine residues).

The polypeptide used in the present invention has the activity of binding to a histone to form a polypeptide aggregate. Herein, in the present description, the term “to form an aggregate” means that the N-terminal domain of PTX3 binds to a histone according to a specific interaction, so as to form a water-insulable dense aggregation state. In addition, in the present description, the term “polypeptide aggregate” means a water-insoluble massive aggregate, comprising the N-terminal domain of PTX3 and a histone.

The histone is one type of protein constituting the chromatin {chromosome} of a eukaryote, and the histone has the activity of binding to DNA. In the present description, the histone is generally derived from vertebrates, is preferably derived front mammals, and is most preferably derived from humans. The histone includes H1, H2A, H2B, H3, and H4. The N-terminal domain of PTX3 and the polypeptide of the present invention bind to generally, at least one type of histone selected from the group consisting of H1, H2A, H2B, In and H4, preferably, at least one type of histone selected from the group consisting of H1, H3 and H4, and more preferably, each of H1, H3 and H4, so as to form a polypeptide aggregate.

The presence or absence of the activity of binding to a histone to form a polypeptide aggregate can be confirmed, for example, by observing the formation of a polypeptide aggregate by visual inspection. For instance, a 1.0 mg/ml histone solution (in a buffer (150 mM NaCl, 20 mM HEPES, 4 mM CaCl₂, and 0.005% surfactant P20 (pH 7.4)) was mixed with an equal amount of a 1.0 mg/ml polypeptide solution as an evaluation target (in the aforementioned buffer). Thereafter, if the presence of a particulate matter can be confirmed by visual inspection, the polypeptide as an evaluation target can be determined to have the activity of binding to a histone to form a polypeptide aggregate. With regard to the observation by visual inspection, if an electronic microscope is further used, the formation of a polypeptide aggregate can be more clearly confirmed.

Otherwise, the presence or absence of the actvity of binding to a histone to form a polypeptide aggregate can also be confirmed by measuring an ultraviolet and visible absorption spectrum. For instance, a 0.1 mg/ml histone solution (in a buffer (150 mM NaCl, 20 mM HEPES, 4 mM CaCl₂, and 0.005% surfactant P20 (pH 7.4)) was mixed with an equal amount of a polypeptide solution as an evaluation target having each concentration (in the aforementioned buffer), and the obtained mixture is then subjected to spectrum measurement if a dose-dependent increase in the absorption spectrum of a UV-visible light (e.g. 310 nm) due to the scattering of an aggregate was observed, or if an increase in the absorption spectrum is observed, when compared with the same concentration of histone alone, the N-terminal domain of PTX3 alone, or the polypeptide of the present invention alone, the polypeptide as an evaluation target can be determined to have the activity of binding to a histone to form a polypeptide aggregate.

Alternatively, the presence or absence of the activity of binding to a histone form a polypeptide aggregate can also be confined by applying immunochromatography. Ouchterlony method, or immunoturbidimetry.

When the formation of a polypeptide aggregate can be confirmed by at least one method among the above-described methods, the polypeptide as an evaluation target can be determined to have the activity of binding to a histone to form a polypeptide aggregate.

In the present invention, there is used a fusion protein of:
(a) at least one polypeptide comprising an amino acid sequence that is identical to or substantially identical to the amino acid sequences of the N-terminal domain of pentraxin 3 capable of binding to a histone to form a polypeptide aggregate: and
(b) the Fe portion of an immunoglobulin.

An immunoglobulin molecule is formed by a disulfide bond (S-S bond) of two heavy chains and two light chains. The heavy chain of an immunoglobulin is constituted with a variable region and a constant region. In mammals, 5 types of constant regions, namely, α, δ, ε, γ, and µ are present, and 5 classes of antibodies are present based on the difference in the constant region (i.e, IgA in the case of α, IgD in the case of δ, IgE in the case of ε, IgG in the case of γ, and IgM in the case of µ). The constant regions α, δ, and γ are each constituted with 3 domains consisting of about 340 amino acids, whereas the constant regions µ and ε are each constituted with 4 domains consisting of about 440 amino acids. The light chain of an immunoglobulin is also constituted with a variable region and a constant region. In mammals, 2 types of light chains, λ (Lambda) and κ (Kappa), are present based on the difference in the constant region. The Fe portion of an immunoglobulin is a portion constituted with the constant regions of a heavy chain and a light chain.

The immunoglobulin may be any one of IgA, IgD, IgE, IgG, and IgM, and the immunoglobulin is preferably IgG. The isotype (subclass) of the immunoglobulin is not particularly limited, either. For example, the isotype of IgG may be any one of IgG1, IgG2a, IgG2b, IgG3, and IgG4.

The amino acid sequences of the Fe regions of the above-described various types of immunoglobulins are known. That is to say, a large number of genes encoding the Fe regions of immunoglobulins have already been isolated and or identified in mammals including humans as typical examples. A large number of the nucleotide sequences thereof have also been reported. For example, the sequence information of nucleotide sequences comprising the Fe regions of human IgG1, IgG2 IgG3, and IgG4 is available from public DNA database such as NCB1, and these nucleotide sequences have been registered under Accession Nos. AJ294730, AJ294731, AJ294732, and AJ294733, respectively. Accordingly, a person skilled in the art could obtain and/or use cDNA encoding such an Fe region portion by designing primers of probes specific to the Fe region, and then applying a common molecular biological method. In this case, with regard to a gene encoding the Fe region to be used, animal species or subtypes are not particularly limited. A gene encoding the Fe region of human IgG1 or IgG2, mouse IgG2a or IgG2b, or the like, which strongly binds to Protein A/G, is preferable.

The binding order of the peptide used in the present invention and the immunoglobulin Fe portion is not particularly limited. As an example, the N-terminus of the immunoglobulin Fe portion can be fused to the C-terminus of the peptide used in the present invention.

The peptide used in the present invention may be fused to the immunoglobulin Fe portion via a linking sequence (linker sequence). As such a linking sequence (linker sequence), an amino acid sequence consisting of, for example, 3 to 20 amino acid residues, preferably 3 to 10 amino acid residues (as an example, GGGGS), can be used,

As a “polypeptide comprising an amino acid sequence that is identical to or substantially identical to the amino acid sequence of the N-terminal domain of pentraxin 3 capable of binding to a histone to form a polypeptide aggregate” (the peptide used in the present invention), only one polypeptide may be used, or two or more polypeptides may also be used.

That is to say, when one peptide used in the present invention is used, a single molecule of the peptide used in the present invention is fused to a single molecule of the Fe portion of an immunoglobulin. On the other hand, when two or more peptides used in the present invention are used, two or more molecules of the peptide used in the present invention are fused to a single molecules of the Fe portion of an immunoglubulin.

The fusion protein of the present invention may be either a free body or a pharmacologically acceptable salt. As such a salt, a salt with a pharmacologically acceptable acid, base, etc. is used, Examples of such a salt may include: salts with inorganic acids (e.g. hydrochloric acid, phosphoric acid, hydrobromic acid, and sulfuric acid), salts with organic acids (e.g. acetic acid, formic acid, propionic acid, fumaric acid, maleic acid, succinic acid, tartaric acid, citric acid, malic acid, oxalic acid, benzoic acid, methanesulfonic acid, and benzenesulfonic acids, alkaline metal salts (e.g. sodium salts and potassium salts), alkaline earth metal salts (e.g. calcium salts and barium salts), magnesium salts, and aluminum salts.

The fusion protein of the present invention is preferably isolated. The term “isolate” means that an operation to eliminate factors other than a component of interest is carried out. The purity of the “isolated polypeptide X” (the percentage of polypeptide X in the weight of all polypeptides) is generally 70% or more, preferably 80% or more, more preferably 90% or more, and most preferably, substantially 100%.

The fusion protein of the present invention can be produced by culturing a transformant, into which an expression vector containing a polynucleotide comprising the nucleotide sequence encoding the fusion protein or a sequence complementary thereto has been introduced, according to a known method, and then seperating the polypeptide from the obtained culture.

The polynucleotide comprising the nucleotide sequence encoding the fusion protein or a sequence complementary thereto may be either DNA or RNA, or may also be a DNA/RNA chimera. The polynucleotide is preferably DNA. In addition, the nucleic acid may be either a double strand or a single strand. In the case of the double strand, it may be double-stranded DNA, double-stranded RNA, or a hybrid of DNA : RNA.

Examples of the DNA comprising the nucleotide sequence encoding the fusion protein of a sequence complementary thereto may include chromosomal DNA, cDNA, synthetic DNA, and a combination thereof. The chromosomal DNA and cDNA encoding the above-described polypeptide can be directly amplified by using, as a template, each of a chromosomal DNA fraction and total RNA or an mRNA fraction prepared from the aforementioned cells or tissues, according to Polymerase Chain Reaction (PCR method) or Reverse Transcriptase-PCR (RT-PCR method). Otherwise, the chromosomal DNA and cDNA encoding the polypeptide used in the present invention can be each cloned from a known chromosomal DNA library and cDNA library prepared by inserting chromosomal DNA, cDNA, and total RNA or mRNA fragments prepared from the aformentioned cells or tissues into a suitable vector, according to a colony or plaque hybridization method, a PCR method, etc.

The fusion protein of the present invention can also be produced according to a known peptide synthesis method. The peptide synthesis method may be either a solid phase synthesis method or a liquid phase synthesis method, A partial peptide or amino acids and a residual portion capable of constituting the fusion protein of the present invention are condensed, and when the obtained product has a protecting group, the protecting group is dissociated, so that a fusion protein of interest can be produced. Condensation of dissociation of a protecting group can be carried out according to known methods.

The thus obtained fusion protein can be purified by a known method. Examples of the puritication method may include solvent extraction, distillation, column chromatography, liquid chromatography, recrystallization, and a combination thereof. In addition, when the polypeptide obtained by the above-described method is a free body, the free body can be converted to a suitable salt (preferably, a pharmacologically acceptable salt) according to a known method or a method equivalent thereto. In contrast, when the polypeptide is obtained in the form of a salt, the salt can be converted to a free body or another salt (preferably, a pharmacologically acceptable salt) according to a known method or a method equivalent thereto.

The fusion protein used in the present invention or a pharmacologically acceptable salt thereof is mixed with a pharmacologically acceptable carrier, as necessary, to prepare a pharmaceutical composition, which can be then used as a therapeutic or preventive agent for infectious disease. By administering a preventively or therapeutically effective amount of the fusion protein of the present invention or a pharmacologically acceptable salt thereof to a mammal, the infections disease of the mammal (preferably, a human) can be prevented or treated. The mammal as an adminstration target is prefeably human, but may also be a mammal other than a human. Examples of such a mammal may include mice, rats, rabbits, dogs, cats, horses, sheep, bovines, goats, pigs, miniature pigs, hairless pigs, and monkeys.

Infectious disease is a generic name for diseases developed by infection with pathogens such as bacteria fungi, viruses, parasites,or abnormal prions. Both diseases that do not exhibit symptoms after infection (inapparent infection), and diseases that exhibit symptoms after infection are collectively referred to as “infectious diseases.” Such infectious disease is developed in various organs in a body, and it mainly causes inflammation in the infected organ, such as, for example, encephalitis, rhinitis, pharyngitis, pneumonia, infective endocarditis, hepatitis, and enteritis. Novel coronavirus infectious disease (COVID-19) is one of such infectious diseases.

Sepsis is a pathogenic condition, in which a severe systemic inflammatory response is provoked by the action of infected microbes and the toxin thereof not only on the infected location, but also on a living body as a whole. Sepsis is a systemic inflammatory response syndrome, and if this disease is not treated, it causes shock, multiple organ failure, disseminated intravascular coagulation (DIC), etc., and thereby, it may lead to death sooner or later. Since sepsis is frequently developed as a complication when immunity has been reduced in a body, it cannot be said the therapeutic results thereof are favorable. The reduced in a body, it cannot be said that the therapeutic results thereof are favorable. The therapeutic of preventive agent for infections disease of the present invention can be preferably used as a therapeutic or preventive agent for sepsis.

There may be a case where acute organ injury is developed in such infectious disease. Examples of the acute organ injury may include acute lung injury, acute kidney injury, acute liver injury, intestinal dysfunction, DIC (disseminated intravascular coagulation), ARDS (acute respiratory distress syndrome), circulatory collapse (septic shock), and septic encephalopathy. The therapeutic or preventive agent for infectious disease of the present invention can be preferably used as a therapeutic or preventive agent for acute organ injury.

In the present invention, as pharmacologically acceptable carriers used for pharmaceutical compositions, various types of organic or inorganic carrier substances, which are commonly used as materials for formulations, are used. Examples of such pharmacologically acceptable carriers may include: carriers for solid formulations, such as excipients, lubricants, binders, and disintegrators: and carriers for liquid formulations, such as solvents, solubilizers, suspending agents, tonicity agents, buffer agents, and soothing agents. Moreover, formulations additives such as antiseptics, antioxidants, coloring agents, and sweeteners can also be used, as necessary. As these carriers, known compounds usable for pharmaceutical compositions can be used, and preferably, commercially available products can be utilized. Furthermore, the amounts of various types of carriers mixed can be determined, as appropriate, by a person skilled in the art.

Examples of the dosage form of the above-described pharmaceutical composition may include: oral agents, such as tablets, capsules (including soft capsules and microcapsules), granules, powder agents, syrup agents, emulsions, and suspending agent; and parenteral agents, such as injections (e.g. subcutaneous injections, intravenous injections, intramuscular injections, intraperitoneal injections, etc.), external agents (e.g. nasal formulations, transdermal formulations, ointments, etc.), suppositories (e.g. rectal suppositories, vaginal suppositories, etc.), pellets, drops, and sustained-release formulations (e.g. sustained-release microcapsules, etc.). These pharmaceutical compositions can be produced according to methods commonly used in the field of formulation technology, for example, the methods described in Japanese Phannacopoeia.

The applied dose of the therapeutic or preventive agent for infectious disease of present invention is preferably an amount sufficient for allowing the fusion protein of the present invention or a pharmacologically acceptable salt thereof to bind to a histone in the body (e.g. in the blood) of a mammal as a therapeutic target to form an aggregate, and for neutralizing the histone. In the case of parenteral administration, the applied dose of the therapeutic or preventive agent of the present invention is different depending on the administration target, the target organ, symptoms, the administration method, etc. The daily dose of the theraputic or preventive agent of the present invention is, for example, about 10 to 1000 mg, preferably about 100 to 500 mg, anti more preferably about 200 to 400 mg, in terms of the weight of the polypeptide of the present invention, with respect to a patient with a body weight of 60 kg. Even when the admimstration target is a mammal other than a human, the dose can be converted to an amount for 60 kg body weight, and can be administered.

The present invention will be described in in the following examples. However, these examples are not intended to limit the scope of the present invention.

### EXAMPLES

### < Example 1 > Production of Construct of PTX3-Fe Fusion Protein, Expression, and Purification

An expression construct of a PTX3-Fc fusion protein, in which the N-terminal (1-50 amino acid residues) peptide of pentraxin 3 (PTX3) was linked to the Fe portion of an immunoglobulin, was produced as fellows. A sequence encoding a PTX partial-length peptide formed by linking GGGGS to the C-terminus consisting of the 50 amino acids of PTX3 (i.e. the 18 to 67 amino acid residues; the 47th and 49th cysteine residues were changed to serine residues) was totally synthesized with a sequence optimized for use in the expression in mammalian cells, and the synthesized sequence was then re-cloned into the Xhol/Spel site of the vector pCAGHyg mlgGl-Fc (Wako),so as to produce an expression vector PTX3(N50)-Fc (FIG. 1a).

Likewise, a sequence encoding a PTX partial-length peptide formed by linking GGGGS to the C-terminus consisting of the 50 amino acids of PTX3 (i.e. a mutant consisting of the 18 to 67 amino acid residues, in which the 47th and 49th cysteine residues were changed to serine residues) was totally synthesized with a sequence optimized for use in the expression in mammalian cells, and the synthesized sequence was then re-cloned into the Xbol/Spel site of the vector pCAG-Hyg mlgGl-Fc (Wako), so as to produce an expression vector PTX3(N50)C5-Fc (FIG. 1b)

Each expression vector was transfected into CHO cells by employing FreeStyle MAX CHO to System (Invitrogen), and then, using Hygromycin B. stable cells were established. From the culture supernatant of each stable cells, individual fusion proteins (PTX3(N50)-Fc and PTX3(N50)CS-Fc) were purified, using 1 mL of HisTrap Protein A (GE Healthcare Life Sciences).

### < Example2 > Supression of Histone Cytotoxicity Against Endothelial Cells by PTX3-Fc Fusion Proteins

Using the above-purified two types of PTX3-Fc fusion proteins (i.e. PTX3(N50)-Fe and PTX3(N50)CS-Fc), the effect of suppressing histone cytotoxicity against vascular endothelial cells (human umbilical vein endothelial cells: HUVEC) was analyzed based on the intake of propidium iodide (PI) into HUVEC.

HUVEC was incubated in an Opti-MEM (Thermo Fisher Scientific) medium, together with the PTX3-Fc fusion protein (PTX3(N50)-Fc or PTX3(N50)CS-Fc) and calf thymus histones (CTH; 50 µg/ml) (Sigma) at 37° C. for 1 hour. Thereafter, PI (10 µg/ml) was added to the resultant, and the obtained mixture was then incubated at 37° C. for 5 minutes. The cells were washed with a phosphate buffier (PBS), were then detached with 1 mM EDTA adjusted with PBS and supplemented with 0.2% PLURONIC (registered trademark) F-68 (Gibeo), and were then analyzed using Guava easyCyte™ Flow Cytometer (Luminex). The degree of cytotoxicity (i.e. the intake level of PI into the cells) was evaluated based on mean fluorescence intensity (MFl).

As a result, it was confirmed that both of the PTX3-Fc fusion proteins, PTX3(N50)-Fc and PTX3(N50)-Fc had the effect of suppressing histone cytotoxicity against vascular endothelial cells (FIG. 2).

### < Example 3 > Improvement of Survival Percentage of Sepsis Mouse Models by PTX3-Fe Fusion Proteins

The PTX3-Fe fusion protein (PTX3(N50)-Fe or PTX3(N50)CS-Fe) was intraperitoneally administered at a dose of 2.7 mg/kg to male C57BL/6 mice, and two hours later, LPS was intraperitoneally administered at a dose of 11 mg/kg thereto. Thereafter, the survival of the mice was observed for 7 days.

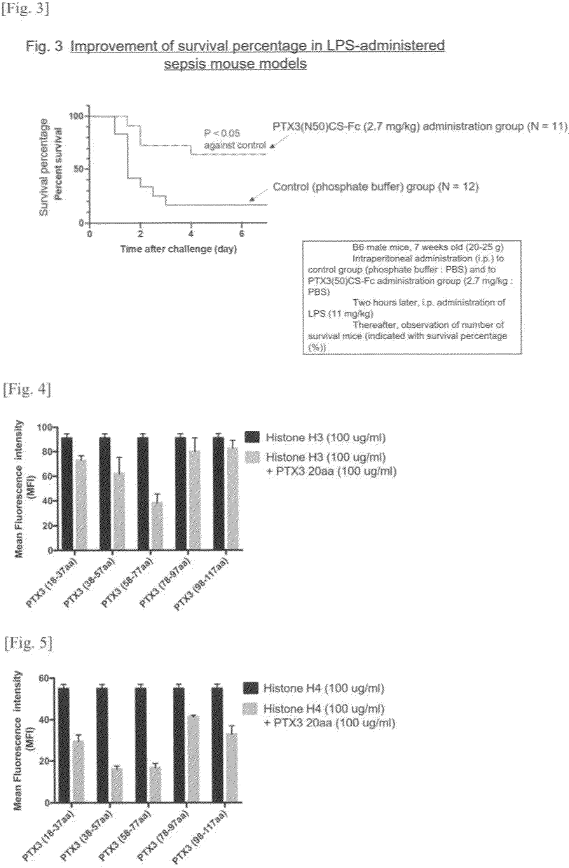

As a result, it was found that the PTX3-Fe fusion protein, PTX3(N50)CS-Fe, significantly improved the survival percentage (FIG. 3).

### < Example 4 > Effects of 20-Amino Acid Peptides of PTX3 on Histone Cytotoxicity Against Vascular Endothelial Cells

Using the 20-amino acid residue (aa) peptides of PTX3 (i.e. PTX3 (18-37 aa). PTX3 (38-57 aa), PTX3 (58-77 aa), PTX3 (78-97 aa), and PTX3 (98-117 aa)), the effects of the 20-amino acid peptides to suppress histone cytotoxicity against HUVEC were analyzed.

HUVEC was incubated in an Opti-MEM (Thermo Fisher Scientific) medium, together with PTX3 20aa (100 µg/ml), recombinant histone H3 (100 µg/ml) (New England BioLab), or recombinant histone H4 (100 µg/ml) (New England BioLab) at 37℃ for 1 hour. Thereafter, PI (10 µg/ml) was added to the resultant, and the obtained mixture was then incubated at 37° C. for 5 minutes. The cells were washed with PBS, were then detached with 1 mM EDTA adjusted with PBS and supplemented with 0.2% PLURONIC (registered trademark) F-68 (Gibco), and were then analyzed using Guava easyCyte™ Flow Cytometer. The degree of cytotoxicity was evaluated based on MFI. The results are shown in FIG. 4 and FIG. 5.

### < Example 5 > Regarding Binding of Spike Protein of SARS-CoV-2 Virus and PTX3

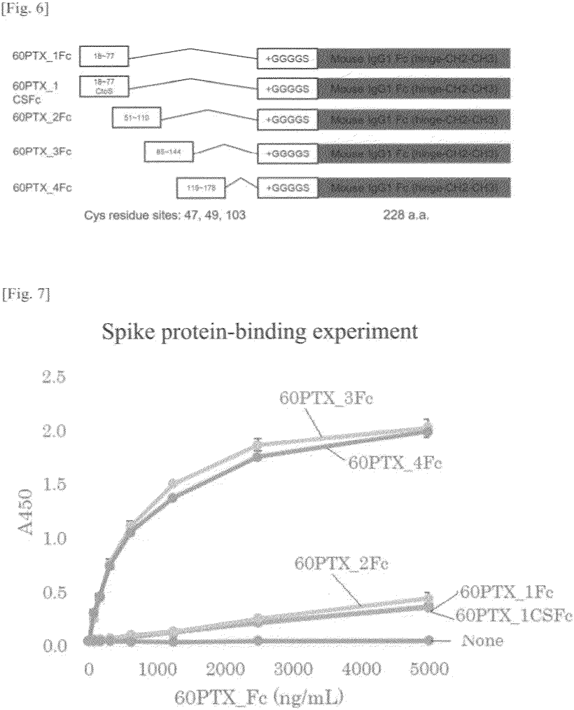

The binding activity of pentraxin 3 (PTX3) to the spike protein (ACROBiosystems; SPN-C52H8) of SARS-CoV-2 virus was measured according to ELISA (FIG. 6 and FIG. 7).

### (Experimental Methods)

### Expression and Purification of Recombinant PTX3

In order to examine the site of the N-terminal protein, which was associated with the binding, 5 types of Fe fusion proteins each having a partial peptide (60 amino acids) were produced.

The cDNA constructs of the PTX3-Fe fusion proteins (60PTX_1Fe, 60PTX_1CSFe, 60PTX_2Fe, 60PTX_3Fe, and 60PTX_4Fe) were produced by totally synthesizing sequences each formed by linking GGGGS to the C-terminus of the 60 amino acids of PTX3 (namely, the amino acid residues 18 to 77; the amino acid residues 18-77 in which the 47th and 49th cysteine residues were mutated to serine residues; the amino acid residues 51 to 110; the amino acid residues 85 to 144; and the amino acid residues 119 to 178), using a codon optimized for use in the expression in mammalian cells, and then re-cloning the obtained sequences into the Xhol/Spel site of the expression vector pCAG-Hyg mlgGI-Fe (Wako). The expression vector was transfected into CHO cells by employing Gibco™ ExpiCHO™ Expression System (Thenno Fisher), and each Fe fusion protein was then purified from the culture supernatant, using Protein A Agarose (GE Healthcare Life Sciences).

FIG. 6 shows the amino acid residue sites of individual PTX3-Fe fusion proteins. 60PTX_ICSFe is a mutant, in which the cysteine residues at positions 47 and 49 were mutated to serine residues.

These 60PTX_Fe fusion proteins were purified from the culture supernatants prepared in a CHO cell expression system, using a Protein A column, and were then used in a binding experiment with a spike protein.

### Binding Assay of Spike Protein With Recombinant 60PTX_Fe Fusion Protein (ELISA)

The spike protein (ACROBiosystems; SPN-C52H8) of SARS-CoV-2 virus was adjusted to a concentration of 2 µg/mL in a Tris buffered saline (TBS)/4 mM CaCl2, and it was then added to a 96-well ELISA plate, so that it was solid-phased at 4℃ overnight. Thereafter, the solution was discarded, and a blocking buffer (TBS/4 mM CaCl2, 0.1% Triton-X100, and 1% BSA) was added to the residue, followed by performing an incubation at room temperature for 2 hours, Thereafter, the reaction mixture was washed with a washing buffer (TBS and 0.1% Triton-X100) 4 times, and a 60PTX_Fe fusion protein, which bad been diluted with a blocking buffer to various types of concentrations, was then added to each well of the plate, followed by performing a reaction at room temperature for 1 hour. The reaction mixture was washed with a washing buffer 4 times, and a detection antibody diluted with a blocking buffer was then added to the resultant, followed by performing a reaction at room temperature for 1 hour. As such a detection antibody for the 60PTX3_Fe fusion, an anti-mouse IgG antibody was used. The reaction mixture was washed with a washing buffer 6 times, and a TMB solution was then added thereto to carry out a coloration reaction for 30 minutes. Then, the absorbance at 450 nm was measured.

### (Results)

The results are shown in FIG. 7. As shown in FIG. 7, 60PTX_Fe and 60PTX_4Fe were observed to have a binding activity to the spike protein.

From the aforementioned results, it is considered that the N-terminus (85 to 178 a.a.) of PTX3 has a site that binds to the spike protein of novel coronavirus. Thus, it is considered that PTX3 directly binds to coronavirus inhibits infection with the coronavirus, and is associated with a defense reaction.

### < Example 6 > Regarding Cytotoxic Action of Nucleocapsid Protein of Cornavirus Against Vascular Endothelial Cells

As a principal protein that constitutes coronavirus, a nucleocapsid (N) protein, as well as the spike (S) protein, has been known. It is considered that the N protein binds to the genornic RNA of the virus to form a ribonucleocapsid, and at the same time, the N protein is a multifunctional protein associated with viral assembly, budding, envelope formation, replication, and regulation of the cell cycle of host cells, translational regulation, inhibition of interferon generation and the like. In addition, it has also been known that a serum N protein is detected in a person infected with SARS-CoV-2 from the early onset, and that a high concentration of anti-N protein antibody, as well as an anti-S protein antibody, is detected in antiserum. From these findings, it is considered that the N protein is deeply associated with the pathological conditions of COVID-19.

Coronavirus is known as a commonly epidemic cold viruses, (HCoV)-HKU1, HCoV-NL63, HCoV-OC43, HCoV-229E and the Hike account for approximately 20%: of common colds, and these coronaviruses exhibit mild symptoms, On the other hand. SARS and MERS caused by SARS-CoV and MERS-CoV have high mortality rates, which are 9% and 38%, respectively. SARS-CoV. 2 is reported to be highly infections among coronaviruses, whereas the mortality rate of COVID-19 varies from 1% or less to 10% or more, depending on area, age, and underlying disease,

In the present example, the cytotoxicity of extracellular histones derived from neutrophils against vascular endothelial cells, and the effects of PTX3 to protect the vascular endothelial cells from such histone cytotoxicity, were mainly observed. The histone is a representative nucleoprotein that interacts with DNA, and it is considered that the N protein of coronavirus also has an action similar to the histone. Thus, the cytotoxic activities of N proteins derived from various types of coronaviruses were measured using human vascular endothelial cells (HUVEC).

### (Experimental Methods)

The measurement performed herein was the same as the assay regarding the histone cytotoxicity against HUVEC, in which the intake of a pigment (propidium iodide, P1) was measured by flow cytometry and an increase in the mean fluorescence intensity (MFI) value was observed. Instead of the histone (whole histone, or H3 or H4), an HCoV-NL69 virus nucleocapsid (N) protein (Human coronavirus (YP_003771.1) (Met1-His377)), a SARS-CoV virus N protein (Human SARS Coronavirus (SARS-CoV) (NP_828858.1) (Met1-Ala422), a SARS-CoV-2 virus N protein (SARS-CoV-2 (2019-nCoV) (YP_009724397.2) (Met1-Ala419) (335Gly/Ala)) (Sino Logical Inc.) were used in each amount of 100 µg/ml.

### (Results)

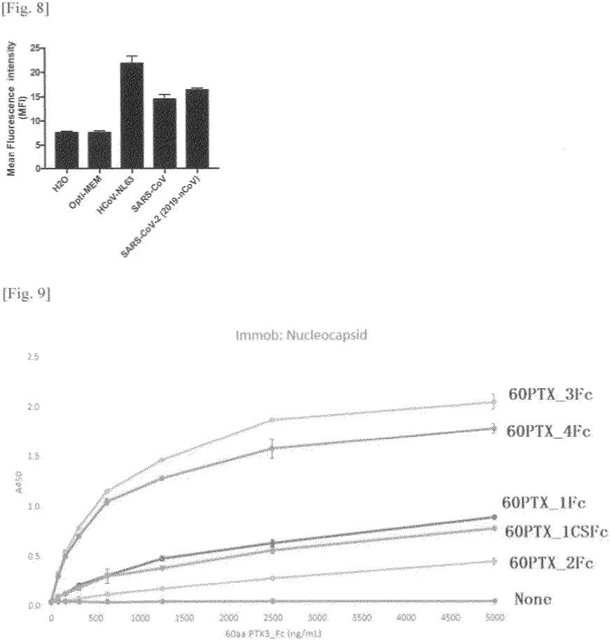

As shown in FIG. 8, when a change in the MFI upon addition of the N protein (100 µg/ml) desired from each coronavirus was observed, a significant increase in the MFI, namely, cytotoxic activity against HUVEC was found upon addition of the N protein. In the present assay, the correlation of virus lethality with an increase to the MFI due to each N protein (i.e. cytotoxic activity) was not found.

Frown the above-described results, it 15 suggested that when the N protein of coronavirus is produced in a large amount and is then released into blood vessels, it would lead to cell damage, and would cause severe pneumonia or vasculitis, and further, organ damage. As such, it is considered that a therapeutic agent capable of preventing coronavirus infection from becoming severe can be provided by inhibiting the N protein, as in the case of the extracellular histone.

### < Example 7 > Suppression of Cytotoxicity of Coronavirus N Protein by PTX3 Partial Peptide

(1) The N protein of SARS-CoV-2, namely SARS-CoV-2 (2019-nCoV) Nucleocapsid-His recombinant Protein (#40588-V08B), was adjusted to a concentration of 2 µg/mL, in a Tris buffer (TBS)/4 mM CaCl₂, and thereafter, it was added in an amount of 50 µl/well to a 96-well ELISA plate, and it was then solid-phased at 4° C. overnight. Thereafter, the solution was discarded, the residue was then washed with a washing buffer (TBS/4 mM CaCl2 0.1% Triton-X100), and a blocking butter (TBS/4 mM CaCl2 0.1% Triton-X100, and 1% BSA) was added to the resultant, followed by performing an incubation at room temperature for 2 hours. Thereafter, the reaction mixture was washed with a washing buffer 4 times, and each type of 60PTX_Fe fusion protein, which had been diluted with a blocking buffer to various types of concentrations, was then added to each well of the plate, followed by performing a reaction at room temperature for 1 hour. Thereafter, the reaction mixture was washed with a washing buffer 4 times, and a detection antibody (HRP-labeled anti-mouse IgG antibody) diluted with a blocking buffer was then added to the resultant, followed by performing a reaction at room temperature for 1 hour. Thereafter, the reaction mixture was washed with a washing buffer 6 times, and a TMB solution was then added thereto to carry out a coloration reaction for 30 minutes. Then, the absorbance at 450 (A450) nm was measured.

### (Results)

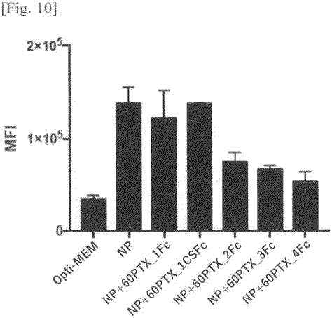

As shown in FIG. 9, 60PTX_1Fe and 60PTX_ICSFe were observed to have a weak binding activity to the SARS-CoV2 virus N protein, whereas 60FTX_3Fe and 60PTX_4Fe fusion protein were observed to have a stronger binding activity to the SARS-CoV2 virus N protein.

From the aforementioned results, it is considered that the N-terminus (85 to 178 a.a.) of PTX3 has a site that binds to the N protein of novel coronaviras. Hence, it is considered that PTX3 directly binds also to the N protein of coronavirus, thus binds to the N proteins exposed from the viral particles or leaked from the cells, eliminates the viruses or the N proteins, and suppresses the cytotoxic action of virus-constituting components such as N proteins or RNA genomes, so that the PTX3 is associated with biological defense against virus attack.

(2) Supression of the cytotoxic activity of the N protein against HUVEC by the 60PTX_Fe fusion proteins purified in Example 5 was examined,

### (Experimental Methods)

The measurement was carried out by measuring the intake of a pigment (PI) according to flow cytometry, and then making a comparison in terms of an increase in the MFI value. HUVEC cells were seeded in an amount of 10,000 cells/well on a 96-well plate, and were then cultured in an Opti-MEM medium (supplemented with 10% FCS) for 2 days. Thereafter, the culture was washed with a phosphate buffer (PBS) twice, and then, only the Opti-MEM medium, the N protem (NP), or NP with each type of 100 µg/ml 60PTX_Fe fusion protein, was added to the resultant culture. As such NP, a 100 µg/ml SARS-CoV-2 virus N protein (SARS-CoV-2 (2019-nCoV) (YP_009724397.2) (Met1-Ala419) (335Gly/Ala)) (Sino Logical Inc.) was used.

The thus obtained mixture was incubated at 37° C. for 1 hour, and P1 (30 µg/ml) was then added to the reaction mixture, followed by performing an incubation for 5 minutes. Thereafter, the reaction mixture was washed with PBS, and the cells were recovered with 1 mM EDTA and 0, 2% PLURONIC and were then measured using a flow cytometer (CyteFlex; Beckman Coulier).

In FIG. 10, MFI indicates Mean Fluorescence Intensity. As shown in FIG. 10, it was found that administration of 60PTX_2Fc, 60PTX_3Fc and 60PTX_4Fc had a significant effect (p< 0.05) of suppressing the cytotoxicity of the N protein.

From the results of the above-described Examples 5 to 7, it is considered that PTX3, in particular, a region of PTX3 comprising the amino acid residues 85 to 178, binds to principal proteins of SARS-CoV-2, namely the 8 protein and the N protein, and is useful for the trapping of the virus, suppression of infection with the virus, and suppression of the infectious disease that becomes severe. That is to say. PTX3 is useful as a therapeutic agent for COVID-19.

### INDUSTRIAL APPLICABILITY

The therapeutic or preventive agent of the present invention is useful in the field of medicaments for treating or preventing infectious disease.

1. A therapeutic or preventive agent for infectious disease, comprising, as an active ingredient, a fusion protein of (a) at least one polypeptide comprising an amino acid sequence that is identical to or substantially identical to the amino acid sequence of the N-terminal domain of pentraxin 3 capable of binding to a histone to form a polypeptide aggregate and (b) the Fc portion of an immunoglobulin, or a pharmacologically acceptable salt thereof.

2. The therapeutic or preventive agent according to claim 1, wherein the amino acid sequence of the N-terminal domain of the pentraxin 3 is a partial sequence consisting of consecutive 15 or more amino acids of the amino acid sequence as set forth in SEQ ID NO: 2.

3. The therapeutic or preventive agent according to claim 1, wherein the amino acid sequence of the N-terminal domain of pentraxin 3 is a partial sequence consisting of consecutive 15 or more amino acids in the amino acid sequence consisting of the 1st to 120th amino acids of the amino acid sequence as set forth in SEQ ID NO: 2.

4. The therapeutic or preventive agent according to claim 1, wherein the amino acid sequence of the N-terminal domain of pentraxin 3 comprises any one of the following regions:
(1) the region consisting of the 18th to 67th amino acids of the amino acid sequence as set forth in SEQ ID NO: 2,
(2) the region consisting of the 18th to 37th amino acids of the amino acid sequence as set forth in SEQ ID NO: 2,
(3) the region consisting of the 38th to 57th amino acids of the amino acid sequence as set forth in SEQ ID NO: 2,
(4) the region consisting of the 58th to 77th amino acids of the amino acid sequence as set forth in SEQ ID NO: 2,
(5) the region consisting of the 78th to 97th amino acids of the amino acid sequence as set forth in SEQ ID NO: 2,
(6) the region consisting of the 98th to 117th amino acids of the amino acid sequence as set forth in SEQ ID NO: 2,
(7) the region consisting of the 85th to 144th amino acids of the amino acid sequence as set forth in SEQ ID NO: 2, and
(8) the region consisting of the 119th to 176th amino acids of the amino acid sequence as set forth in SEQ ID NO: 2.

5. The therapeutic or preventive agent according to claim 2, wherein at least one cysteine residue of the amino acid sequence as set forth in SEQ ID NO: 2 is substituted with another amino acid residue.

6. The therapeutic or preventive agent according to claim 2, wherein the cysteine residues that are the 47th and 49th amino acid residues of the amino acid sequence as set forth in SEQ ID NO: 2 are substituted with other amino acid residues.

7. The therapeutic or preventive agent according to claim 5, wherein the other amino acid residues are serine residues.

8. The therapeutic or preventive agent according to claim 1, wherein (b) the N-terminus of the immunoglobulin Fc portion is fused to the C-terminus of (a) the polypeptide comprising an amino acid sequence that is identical to or substantially identical to the amino acid sequence of the N-terminal domain of pentraxin 3 capable of binding to a histone to form a polypeptide aggregate.

9. The therapeutic or preventive agent according to claim 1, wherein the infectious disease is sepsis.

10. The therapeutic or preventive agent according to claim 1, wherein the infectious disease is COVID-19 infection.


SEQUENCE LISTING (3 sequences)

SEQ ID NO: 1 - DNA, 1908 bp, Artificial
  Histone H3 fragment
  CDS (117)..(1262)
catttattaaggactctctgctccagcctctcactctcactctcctccgctcaaactcagctcacttgagagtctcctcccgccagctgtggaaagaactttgcgtctctccagcaatgcatctccttgcgattctgttttgtgctctctggtctgcagtgttggccgagaactcggatgattatgatctcatgtatgtgaatttggacaacgaaatagacaatggactccatcccactgaggaccccacgccgtgcgactgcggtcaggagcactcggaatgggacaagctcttcatcatgctggagaactcgcagatgagagagcgcatgctgctgcaagccacggacgacgtcctgcggggcgagctgcagaggctgcgggaggagctgggccggctcgcggaaagcctggcgaggccgtgcgcgccgggggctcccgcagaggccaggctgaccagtgctctggacgagctgctgcaggcgacccgcgacgcgggccgcaggctggcgcgtatggagggcgcggaggcgcagcgcccagaggaggcggggcgcgccctggccgcggtgctagaggagctgcggcagacgcgagccgacctgcacgcggtgcagggctgggctgcccggagctggctgccggcaggttgtgaaacagctattttattcccaatgcgttccaagaagatttttggaagcgtgcatccagtgagaccaatgaggcttgagtcttttagtgcctgcatttgggtcaaagccacagatgtattaaacaaaaccatcctgttttcctatggcacaaagaggaatccatatgaaatccagctgtatctcagctaccaatccatagtgtttgtggtgggtggagaggagaacaaactggttgctgaagccatggtttccctgggaaggtggacccacctgtgcggcacctggaattcagaggaagggctcacatccttgtgggtaaatggtgaactggcggctaccactgttgagatggccacaggtcacattgttcctgagggaggaatcctgcagattggccaagaaaagaatggctgctgtgtgggtggtggctttgatgaaacattagccttctctgggagactcacaggcttcaatatctgggatagtgttcttagcaatgaagagataagagagaccggaggagcagagtcttgtcacatccgggggaatattgttgggtggggagtcacagagatccagccacatggaggagctcagtatgtttcataaatgttgtgaaactccacttgaagccaaagaaagaaactcacacttaaaacacatgccagttgggaaggtctgaaaactcagtgcataataggaacacttgagactaatgaaagagagagttgagaccaatctttatttgtactggccaaatactgaataaacagttgaaggaaagacattggaaaaagcttttgaggataatgttactagactttatgccatggtgctttcagtttaatgctgtgtctctgtcagataaactctcaaataattaaaaaggactgtattgttgaacagagggacaattgttttacttttctttggttaattttgttttggccagagatgaattttacattggaagaataacaaaataagatttgttgtccattgttcattgttattggtatgtaccttattacaaaaaaaatgatgaaaacatatttatactacaaggtgacttaacaactataaatgtagtttatgtgttataatcgaatgtcacgtttttgagaagatagtcatataagttatattgcaaaagggatttgtattaatttaagactatttttgtaaagctctactgtaaataaaatattttataaaactaaaaaaaaaaaaaaaaaaaaaaaaaaaaaaaaaaaaa

SEQ ID NO: 2 - protein, 381 aa, Artificial
  Synthetic Construct
MHLLAILFCALWSAVLAENSDDYDLMYVNLDNEIDNGLHPTEDPTPCDCGQEHSEWDKLFIMLENSQMRERMLLQATDDVLRGELQRLREELGRLAESLARPCAPGAPAEARLTSALDELLQATRDAGRRLARMEGAEAQRPEEAGRALAAVLEELRQTRADLHAVQGWAARSWLPAGCETAILFPMRSKKIFGSVHPVRPMRLESFSACIWVKATDVLNKTILFSYGTKRNPYEIQLYLSYQSIVFVVGGEENKLVAEAMVSLGRWTHLCGTWNSEEGLTSLWVNGELAATTVEMATGHIVPEGGILQIGQEKNGCCVGGGFDETLAFSGRLTGFNIWDSVLSNEEIRETGGAESCHIRGNIVGWGVTEIQPHGGAQYVS

SEQ ID NO: 3 - protein, 161 aa, Artificial
  Histone H3 fragment
ENSDDYDLMYVNLDNEIDNGLHPTEDPTPCDCGQEHSEWDKLFIMLENSQMRERMLLQATDDVLRGELQRLREELGRLAESLARPCAPGAPAEARLTSALDELLQATRDAGRRLARMEGAEAQRPEEAGRALAAVLEELRQTRADLHAVQGWAARSWLPAG